(19) United States
(12) Patent Application Publication
(10) Pub. No.: US 2020/0261726 A1
(43) Pub. Date: Aug. 20, 2020

(54) Conductive Polymer Implant, combining electrical and chemical stimulation to improve neural recovery

(71) Applicant: The Board of Trustees of the Leland Stanford Junior University, Stanford, CA (US)

(72) Inventors: Byeongtaek Oh, Columbia, MD (US); Alexa Levinson, Mountain View, CA (US); Paul George, Sunnyvale, CA (US)

(21) Appl. No.: 16/648,049

(22) PCT Filed: Oct. 4, 2018
(86) PCT No.: PCT/US2018/054455

§ 371 Date: Mar. 17, 2020

Related U.S. Application Data
(60) Provisional application No. 62/568,767, filed on Oct. 5, 2017.

(51) Int. Cl. A61N 1/36; A61K 35/28; A61N 1/05; A61N 1/04

(52) U.S. Cl. CPC A61N 1/36121; A61N 1/0464; A61N 1/0531; A61K 35/28

(57) ABSTRACT

Improved in vivo brain therapy is provided with a system having a neural implant that delivers both electrical stimulation and stem cell therapy to the brain. The return electrode for electrical stimulation is spaced apart from the implant to prevent local short-circuiting of the electrical stimulation. After forming the implant, stem cells can be seeded upon it, and subsequently, the apparatus can be implanted in vivo. A cannula system allows for continued electrical stimulation and the ability to manipulate the stem cells within the host environment.

### FIELD OF THE INVENTION

This invention relates to neural implants.

### BACKGROUND

The development of new conductive biocompatible implants for use in medicine is a significant issue in biomedical engineering. The main requirement is to design an implant that mimics the biological and mechanical properties with human tissues and allows for continued interactions with the biological system. However, the limited electrical conductivity of most implants and lack of mobility from an in vitro cell culture to an in vivo system restricts previous applications for rehabilitation.

### SUMMARY

To resolve this problem, a conductive polymer implant has been formed to electrically stimulate stem cells. After forming the implant, stem cells can be seeded upon it, and subsequently, the apparatus can be implanted in vivo. A cannula system allows for continued electrical stimulation and the ability to manipulate the stem cells within the host environment. It is therefore an object of this work to provide a conductive polymer implant attached to a cannula, which allows us to manipulate the cells in both an in vitro culture and an in vivo stimulation.

This work considers a biocomposite of a functionalized polymer implant and its use for electrically stimulating cells in vivo to help with neural tissue engineering applications, such as neural network regeneration and neural augmentation. It also emphasizes processes for preparing the conductive polymer implant and introduces the concept of combining electrical stimulation with stem cell therapies to improve neural recovery, specifically in stroke applications.

Various applications are possible. The improved versatility of a biocompatible conductive polymer implant attached via a cannula system allows for a wider arrange of in vivo applications compared to just a cannula or implant alone. For example, the polymer implant with cannula allows us to specifically target stem cell treatment to the region of interest. In addition, stimulating the cells in vivo allows for the release of various paracrine factors directly onto the desired region. This system allows for combined electrical stimulation and chemical stimulation (from the stem cells) to improve neural recovery, a method which has not been demonstrated previously. The polymer implant has multiple potential biomedical applications because of its biocompatibility. Moreover, the addition of a cannula combined with the high electrical conductivity of the polymer allows the use of electrical stimulation in vivo for controlling the differentiation and paracrine release of stem cells, which maximizes the utilization of stem cells for neural recovery.

Significant advantages are provided. Stem cells can target brain repair and have a therapeutic effect on the patient months or even years post-injury. Stem cells can be used as treatment options for various brain diseases including stroke, Alzheimer's disease, and glioblastoma. However, ineffective delivery of transplanted cells to the ischemic site is a major hurdle hampering the clinical application of human neuronal progenitor cells (hNPCs)-based stroke therapy. One of the main challenges in stem cell transplantation is to minimize cell death after implantation and maintain electrical interactions with the cells after seeding. With the use of a conductive polymer to provide an appropriate stem cell niche, the hNPCs can be transplanted into human brain to help restore function after stroke in the near future. Because we can stimulate the stem cells after transplantation, we can also isolate important repair mechanisms, which could lead to novel drug therapies for stroke recovery. The improved versatility of the combination of an electrically conductive polymer plate attached to a cannula provides an implantable and novel way to create a new paradigm to augment stem cell-induced brain disease treatments as well as a new method of stem cell delivery. The application of electrical and chemical stimuli (from the cells) provides a new paradigm to improve neural recovery.

### BRIEF DESCRIPTION OF THE DRAWINGS

FIGS. 1A-B show an exemplary embodiment of the invention.

FIG. 2 shows stroke recovery results in rat experiments for various therapy approaches.

FIG. 3 is rat brain imaging results showing improved results (more specifically increased endogenous repair mechanisms) from combined stem cell and electrical stimulation therapy.

FIG. 4 is quantitative results showing improved results from combined stem cell (SC) and electrical stimulation (ES) therapy.

FIG. 5 is a heat map showing differences in gene expression between ES+SC therapy and SC therapy.

FIG. 6 is a scatter plot for RNA-sequencing that demonstrates that ES+SC therapy causes different gene expressions than SC therapy.

FIG. 7 shows quantitative real-time PCR (qRT-PCR) results from several different therapies.

### DETAILED DESCRIPTION

### Introduction

Stroke is a leading cause of death and disability in the United States. Despite biomedical advancements in clinical trials, no medical therapies exist for stroke outside the acute time window. Due to the severity and prevalence of stroke, identifying novel and effective therapies is important for helping stroke survivors. Our previous study revealed that in vitro electrical stimulation enhanced stem cells' efficacy on stroke recovery.

Brain stimulation techniques that enhance stroke recovery are a promising approach of research; however, in vivo electrical stimulation in combination with neural progenitor cell transplantation has not been fully investigated. To understand the efficacy of stem cell therapy and mechanisms driving recovery, we describe the use of a cannula implant including a conductive polypyrrole (PPy) and reference electrode to allow for continued stimulation of transplanted cells in order to maximize stem cell-based stroke therapeutics.

The polymeric cannula system is uniquely configured so that it can be fixed to the skull for electrical attachments and also positioned on the brain surface for stem cell delivery as described in more detail below. The placement of the electrical connections separated on the skull from the stem cell-seeded conductive polymer insures there is no incidental electrical communication and forces the electrical signal to be between the conductive polymer scaffold and the reference electrode. The reference electrode is preferably placed on the opposite side of the skull to force the electrical field through the brain tissue and seeded-stem cells. This is the first system that will allow for combined chemical signaling (through the factors produced from the stem cells and/or factors seeded in the polymer) and electrical stimulation to improve recovery. This more accurately creates an environment for recovery similar to the developing nervous system environment where chemical, physical and electrical cues help form connections and neural circuits. The system configuration allows for subjects to perform rehabilitation activities or other normal activities while being stimulated which will help strengthen remaining pathways after injury. Prior devices have concentrated on delivering stem cells or electricity but have not focused on delivering both signals in coordination. Our research has shown that the combined effects of electrical stimulation and chemical stimulation (via the seeded stem cells) increase endogenous stem cells production which is known to correlate with improved recovery. This work allows for the adjustment of various parameters (e.g. density of cells, electrical stimulation) to target the increase of endogenous stem cells to improve neural recovery. Finally, we have seen that alternating current (AC) forms of stimulation are able to be delivered and are more effective than DC stimulation patterns.

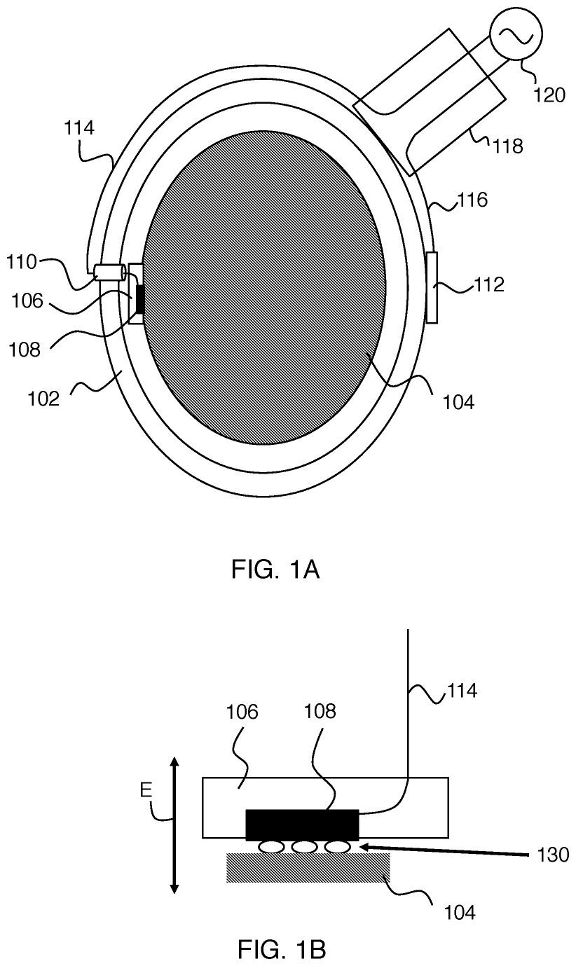

FIGS. 1A-B show an exemplary embodiment of the invention. Here FIG. 1B is an enlarged side view of neural implant 106 of FIG. 1A. This embodiment is an apparatus for providing in vivo neural therapy including:

i) a neural implant 106 configured to simultaneously provide in vivo electrical stimulation to the brain 104 of a subject and stem cell therapy to the brain of the subject (e.g., with stem cells 130 disposed on a polymer scaffold 108);

ii) a reference electrode 112 disposed on a head 102 of the subject at a reference location spaced apart from an implant location of the neural implant 106; and

iii) an electrical connection unit 118 affixed to the head 102 of the subject and electrically connected to the neural implant and to the reference electrode (via insulated wires 114 and 116 respectively), where the electrical connection to the neural implant 106 is via a cannula 110 through the skull of the subject, as shown. Having the return electrode for electrical stimulation spaced apart from the implant improves effectiveness of electrical stimulation by preventing it from locally short-circuiting at the implant location.

The following features of preferred embodiments can be practiced individually or in any combination.

The reference location is preferably substantially opposite the implant location relative to the head of the subject, as shown on FIG. 1A. The stem cell therapy can include providing chemical signals to the brain of the subject with stem cells in the neural implant. The in vivo electrical stimulation is preferably an AC electrical stimulation preferably having a frequency in a range from 1 Hz to 300 Hz. The neural implant can be configured to release one or more chemical agents to the brain of the subject in vivo, e.g., by leaching out from the polymer scaffold over time, or in a pulsed release triggered with an electrical control signal or by the electrical stimulation.

Practice of the invention does not depend critically on the material composition of the scaffold 108. In the experimental example described below, electroplated-polypyrrole (PPy) is the material employed, but any scaffold capable of holding the stem cells in the neural implant can be employed. Practice of the invention also does not depend critically on the kind of stem cells employed. For simplicity of description, ‘stem cells’ is taken here to include both unrestricted stem cells and restricted stem cells such as neural progenitor cells. Practice of the invention also does not depend critically on the electronics used to drive the implant. FIG. 1A shows a generic electrical source 120 for this, but any electrical circuit or system capable of driving the implant as needed can be employed.

The neural implant 106 preferably includes a polymer scaffold 108 configured to hold living stem cells 130 for the stem cell therapy. The neural implant can be configured to provide in vitro electrical stimulation to the living stem cells prior to being disposed on the brain of the subject. In this way, electrical stimulation to the stem cells can be provided both in vitro and later on in vivo without ever needing to reform new electrical connections to the stem cells for the in vivo stimulation.

The in vivo electrical stimulation and stem cell therapy are preferably configured to promote endogenous stem cell production. Experimental examples of this capability are described below.

### Experimental Demonstration

Experiments as described in the methods section below were carried out on lab animals, with the following results.

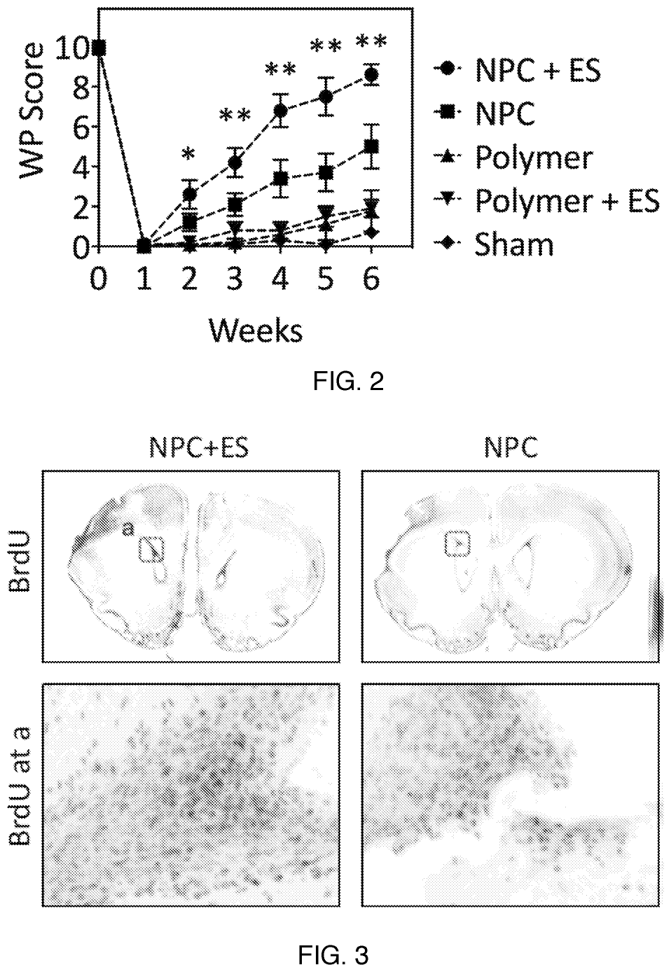

FIG. 2 shows that electrical stimulation of NPC (neural progenitor cells) using the cannula system of this work augments functional recovery after stroke. (* and ** indicate statistically significant differences between groups, p<0.05 and 0.01, respectively). Here sham is the control (scaffold only with no cells or stimulation), polymer is scaffold only (no stem cells), polymer+ES is scaffold only+in vivo electrical stimulation, NPC is scaffold+stem cells, and NPC+ES is scaffold+stem cells+in vivo electrical stimulation.

FIG. 3 shows images that demonstrate that electrical stimulation (left side of figure)+NPCs increases endogenous stem cell (BrdU+) population in subventricular zone (SVZ) relative to NPCs alone (right side of figure). The black dashed square (a) in the top left indicates the SVZ, while the bottom left is an enlarged view of region (a). Similarly, the bottom right of the figure is an enlarged view of the boxed region of the upper right part of the figure. Here BrdU is short for Bromodeoxyuridine, which is widely used in the detection of proliferating cells in living tissue.

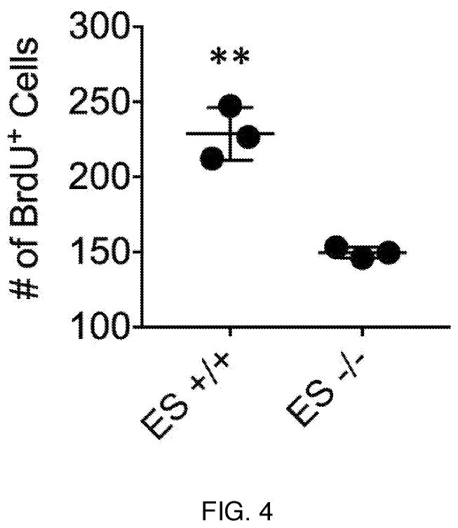

FIG. 4 show the quantification of the number of BrdU+ cells in the SVZ. Electrical stimulation augments the number of cells positive to BrdU. Here ES+/+ refers to NPC+stimulation therapy and ES−/− refers to NPC therapy alone.

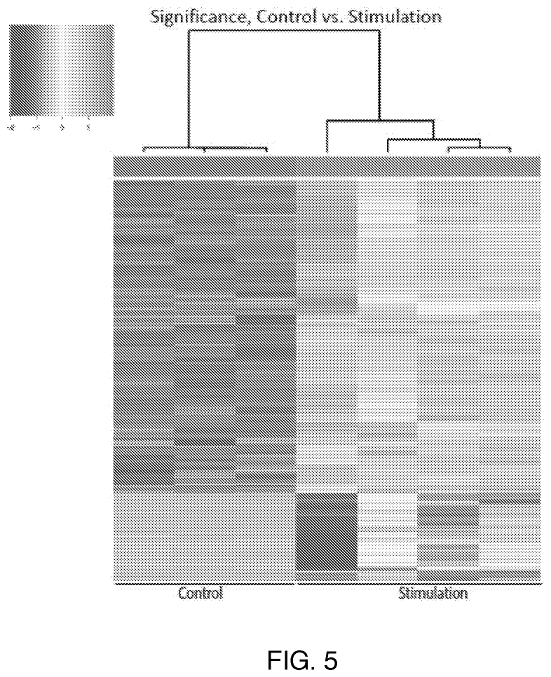

Further experiments on combined electrical and stem cell stimulation not related to the above animal experiments have also been performed. FIGS. 5-7 relate to stimulation experiments performed in vitro.

FIG. 5 is a heatmap analysis demonstrating that electrical stimulation affects transcriptome changes and causes different gene expressions. Here ‘control’ refers to NPC-only therapy and ‘stimulation’ refers to NPC+electrical stimulation therapy.

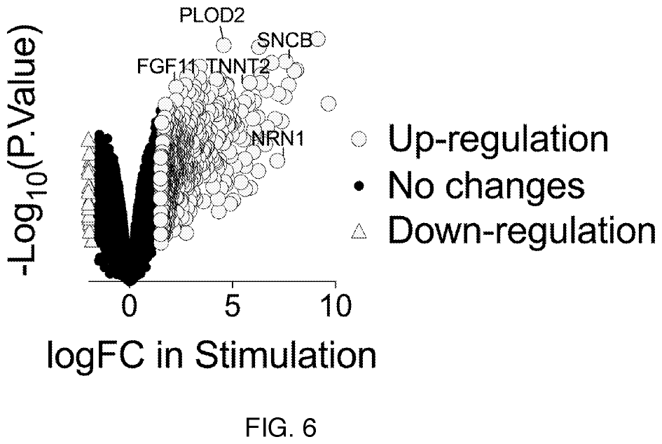

FIG. 6 is a volcano plot representing the transcriptome changes in stem cells in vitro after the stimulation. A large population of genes has been up-regulated by the stimulation. Due to a large variation in RNA-seq technique, we operated qRT-PCR analysis to cross-validate the findings from the sequencing. It showed that STC2 (Stanniocalcin 2), up-regulated by the stimulation was highly produced by stem cells after the stimulation. Here PLOD2 is short for ‘Procollagen-Lysine, 2-Oxoglutarate 5-Dioxygenease 2’, FGF11 is short for Fibroblast growth factor 11, TNNT2 is short for Troponin, NRN1 is short for Neuritin 1 and SNCB is short for Synuclein Beta.

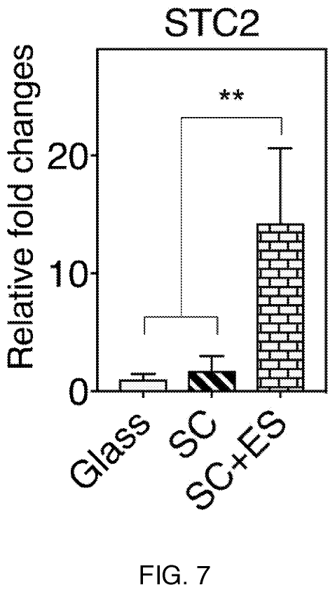

FIG. 7 shows the quantitative real-time PCR (qRT-PCR) analysis of STC2. The electrical stimulation+SC (stem cells) induced much larger STC2 gene expression as compared to the cells cultured on glass and SCs without the stimulation.

### Materials and Methods

The cannula implant wired with electroplated-polypyrrole (PPy) and reference electrode (stainless steel mesh, 0.25 cm²) was designed to deliver human neural progenitor cells (NPCs, Aruna Biomedical) with in vivo electrical stimulation (FIG. 1a). Animals (male T-cell deficient nude rats (NIH-RNU 230±30 g)) were trained 3 times before baseline. After baseline, the animals underwent dMCA (distal middle cerebral artery) occlusion and were tested 1 week post-stroke prior to implantation (animals without a significant deficit (>30% of baseline) were removed). One week after stroke, animals were randomized by vibrissae-whisker paw score, and the cannula implantation surgeries performed by a blinded individual. Electrical stimulation (AC: ±400 mV/100 Hz for 1 hr, 1 day after implantation, n=5-10) was applied daily for 1 hr on three consecutive days. Behavior testing after the cannula surgeries performed by blinded individuals. After 6 weeks post-stroke, rats were perfused and 40 μm coronal slices were sectioned and incubated overnight at 4° C. (anti-BrdU, connective tissue growth factor (1:100), Abcam). Images were analyzed on a Keyence microscopy with ImageJ software.

1. Apparatus for providing in vivo neural therapy, the apparatus comprising:
a neural implant configured to simultaneously provide a) in vivo electrical stimulation to a brain of a subject, and b) stem cell therapy to the brain of the subject;
a reference electrode disposed on a head of the subject at a reference location spaced apart from an implant location of the neural implant; and
an electrical connection unit affixed to the head of the subject and electrically connected to the neural implant and to the reference electrode, wherein the electrical connection to the neural implant is via a cannula through a skull of the subject.

2. The apparatus of claim 1, wherein the reference location is substantially opposite the implant location relative to the head of the subject.

3. The apparatus of claim 1, wherein the neural implant includes a polymer scaffold configured to hold living stem cells for the stem cell therapy.

4. The apparatus of claim 3, wherein the neural implant is configured to provide in vitro electrical stimulation to the living stem cells prior to being disposed on the brain of the subject.

5. The apparatus of claim 1, wherein the in vivo electrical stimulation and stem cell therapy are configured to promote endogenous stem cell production.

6. The apparatus of claim 1, wherein the stem cell therapy comprises providing chemical signals to the brain of the subject with stem cells in the neural implant.

7. The apparatus of claim 1, wherein the in vivo electrical stimulation is an AC electrical stimulation.

8. The apparatus of claim 6, wherein the AC electrical stimulation has a frequency in a range from 1 Hz to 300 Hz.

9. The apparatus of claim 1, wherein the neural implant is configured to release one or more chemical agents to the brain of the subject in vivo.
